(19) United States
(12) Patent Application Publication
(10) Pub. No.: US 2026/0251576 A1
(43) Pub. Date: Aug. 27, 2026

(54) METHOD AND DEVICE FOR SAMPLING AND IDENTIFICATION OF MATERIALS

(71) Applicant: The Government of the United States of America, as represented by the Secretary of Homeland Security, Washington, DC (US)

(72) Inventors: Jeffrey Brian Barber, Vineland, NJ (US); Benjamin Paul Wilkins, Tabernacle, NJ (US)

(73) Assignee: The Government of the United States of America, as represented by the Secretary of Homeland Security, Washington, DC (US)

(21) Appl. No.: 19/539,839

(22) Filed: Feb. 13, 2026

Related U.S. Application Data
(60) Provisional application No. 63/761,656, filed on Feb. 21, 2025.

(51) Int. Cl. G01N 21/65; B01L 3/00; G01N 1/12; A61B 17/22

(52) U.S. Cl. CPC G01N 21/65; B01L 3/50825; G01N 1/12; G01N 21/658; A61B 2017/22082

(57) ABSTRACT

In an example, a material sampling and identification device includes a bulbous member including a bulbous cavity at a closed end; and an elongated tube having a connection end coupled to the bulbous member and including a hollow interior which extends from the bulbous cavity of the bulbous member through the connection end to an open end which is configured as a scoopula end.

### CROSS-REFERENCE TO RELATED APPLICATIONS

The application is a nonprovisional of and claims the benefit of priority from U.S. Provisional Patent Application No. 63/761,656, filed on Feb. 21, 2025, entitled METHOD AND DEVICE FOR SAMPLING AND IDENTIFICATION OF MATERIALS, the disclosure of which is incorporated by reference in its entirety.

### STATEMENT OF GOVERNMENT INTEREST

This invention was made with Government support, provided by the United States Department of Homeland Security, and by an employee of the United States Department of Homeland Security in the performance of their official duties. The U.S. Government has certain rights in this invention.

### FIELD

The discussion below relates generally to methods and devices for sampling and identification of materials and, more particularly, to the construction and use of a sampling device that is configured and adaptable for different material interrogation techniques.

### BACKGROUND

The sampling and identification of materials refers to the process of taking a representative portion (sample) from a larger material to analyze its composition and determine its exact type, often employing techniques such as spectroscopy, to verify its identity and quality. The technology can be used for quality control or security screening purposes, for instance.

### SUMMARY

Embodiments of the present invention are directed to a versatile and robust design of a sampling and identification device that is configured and adaptable for different material interrogation techniques.

In accordance with an aspect of the present disclosure, a material sampling and identification device comprises a bulbous member including a bulbous cavity at a closed end; and an elongated tube having a connection end coupled to the bulbous member and including a hollow interior which extends from the bulbous cavity of the bulbous member through the connection end to an open end which is configured as a scoopula end.

Another aspect of the disclosure is directed to a method for sampling a target material using a sampling device which includes a bulbous member having a bulbous cavity at a closed end and an elongated tube having a connection end coupled to the bulbous member and including a hollow interior which extends from the bulbous cavity of the bulbous member through the connection end to an open end which is configured as a scoopula end. The method comprises introducing the target material via the open end of the elongated tube through the hollow interior into the bulbous cavity of the bulbous member of the sampling device; and interrogating the target material in the sampling device.

In accordance with another aspect of this disclosure, a material sampling and identification system comprises a bulbous member including a bulbous cavity at a closed end; an elongated tube having a connection end coupled to the bulbous member and including a hollow interior which extends from the bulbous cavity of the bulbous member through the connection end to an open end; and a sampling stand having a concave interior to receive the bulbous member and position the elongated tube above the bulbous member to keep a target material contained within the bulbous cavity.

Other features and aspects of various examples and embodiments will become apparent to those of ordinary skill in the art from the following detailed description which discloses, in conjunction with the accompanying drawings, examples that explain features in accordance with embodiments. This summary is not intended to identify key or essential features, nor is it intended to limit the scope of the invention, which is defined solely by the claims.

### BRIEF DESCRIPTION OF THE DRAWINGS

The attached drawings help explain the embodiments described below.

FIG. 1 is an elevational view of a material sampling and identification device.

FIG. 2 shows the material sampling and identification device supported on a sampling stand for interrogation of a target material.

FIG. 3 shows the material sampling and identification device supported on the sampling stand for interrogation of the target material using another technique.

FIG. 4 shows an example of a mounting device for mounting the sampling stand.

FIG. 4A shows a blunted cone-shaped mount as an example of a wedge mount.

FIG. 4B shows a blunted pyramid-shaped mount as another example of a wedge mount.

FIG. 4C shows a blunted wedge-shaped mount as another example of a wedge mount.

FIG. 5 is a close-up view of the mounting device of FIG. 4.

FIG. 6 is a schematic view illustrating an example of a containment tray.

FIG. 7 shows an example of a flow diagram illustrating a method for sampling a target material using the sampling device.

### DETAILED DESCRIPTION

A number of examples or embodiments of the present invention are described, and it should be appreciated that the present invention provides many applicable inventive concepts that can be embodied in a variety of ways. The embodiments discussed herein are merely illustrative of ways to make and use the invention and are not intended to limit the scope of the invention. Rather, as will be appreciated by one of skill in the art, the teachings and disclosures herein can be combined or rearranged with other portions of this disclosure along with the knowledge of one of ordinary skill in the art.

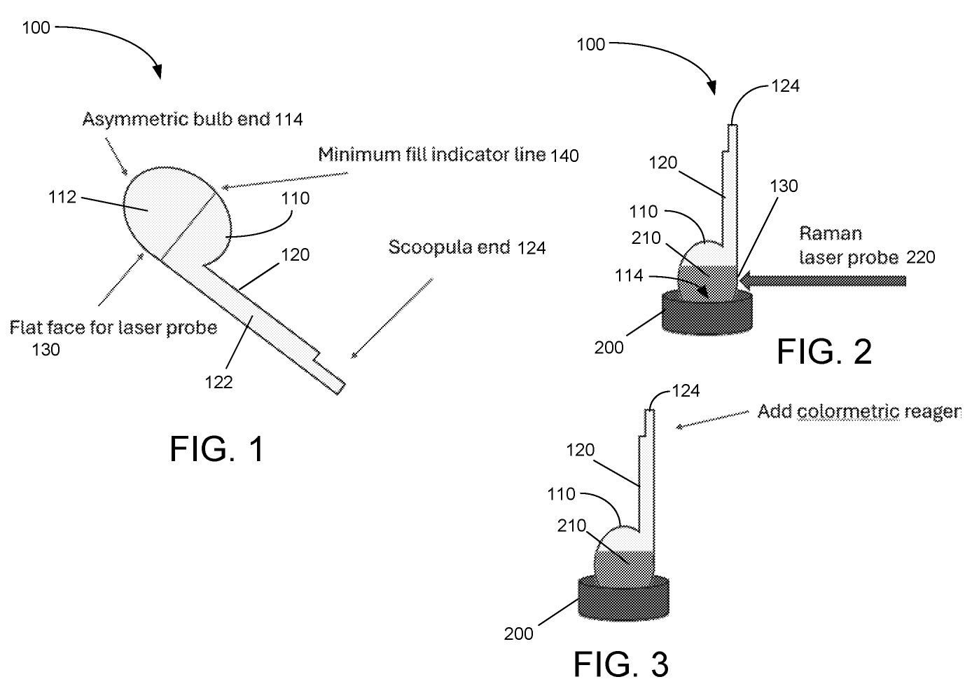

FIG. 1 is an elevational view of a material sampling and identification device 100 or a sampling device for brevity. The sampling device 100 can be used as an alarm resolution sampling tool (ARST). It includes a bulbous member 110 with a bulbous cavity 112 at a closed end 114 and an elongated tube 120 having a connection end coupled to the bulbous member 110 and including a hollow interior 122 which extends from the bulbous cavity 112 of the bulbous member 110 through the connection end to an open end 124 on the opposite side of the elongated tube 120. The open end 124 may be configured as a scoopula end 124. The elongated tube 120 may be centered in the body of the bulbous member 110. Alternatively, as seen in FIG. 1, the connection end of the elongated tube 120 is coupled to the bulbous member 110 at an offset location of the bulbous member 110 to be offset to one side relative to the bulbous member 110. The bulbous member 110 may be symmetrical or asymmetrical. In the asymmetrical bulbous member 110 as shown, the overall shape of the bulbous member 110 is generally round with a flat or planar side surface 130 on a lateral side. Shapes that are generally round include spherical, ellipsoidal, oblong, etc. The planar side surface 130 is parallel to a longitudinal axis of the elongated tube 120. The flat or planar surface 130 in a cross-sectional dimension may be configured to make contact with a laser probe such as a Raman laser probe (e.g., in lateral contact with probe 220 as seen in FIG. 2). In other embodiments, the bulbous member 110 may be curved/circular in the same cross-sectional dimension.

The bulbous member 110 and/or the elongated tube 120 may include one or more graduation marks as one or more fill indicators including, for example, a minimum fill indicator line 140 to ensure a sufficient amount of sample is captured before it is analyzed and identified. The graduation marks are spaced along a longitudinal axis of the elongated tube 120. These indicators may be general (e.g., 1 mL increments) or specific to certain interrogation techniques (e.g., powder fill level for interrogation with a particular Bulk Resolution System or liquid fill level for colorimetric reagent addition).

The material sampling and ID device 100 enables and facilitates the sampling and containment of liquids and solids (e.g., powders) for purposes of interrogating with optical (e.g., Raman spectroscopy) and/or chemical (e.g., colorimetric) methods in order to detect potential threat materials (e.g., explosives, drugs, biothreats, etc.).

The sampling device 100 has a configuration similar to a disposable transfer pipette, with the bulbous end as a closed end 114 of the bulbous member 110 connected to the elongated tube 120 which extends to the scoopula opening 124. A user may squeeze the bulb or bulbous member 110 to suck or draw a sample or target material in the form of liquids or powders into the bulbous interior or cavity 112 via the elongated tube 120. In the embodiment shown, the elongated tube 120 has the scoopula end 124. In another embodiment, the elongated tube 120 can be trimmed by tearing off the tube at pre-formed sections of thinner material designed to tear more easily along a tube diagonal to enable the elongated tube 120 to be used as a scoopula. In yet another embodiment, the elongated tube diameter may vary linearly along its length or in a stepwise manner, decreasing in lateral size from the connection end (coupled to the bulbous member 110 at the closed end 114) to the open end 124.

Rather than ejecting the liquid/powder (normal operation for a transfer pipette), the bulbous cavity 112 of the sampling device 100 becomes storage for the sample or target material. Storing the material of interest in the bulbous cavity 112 allows the material to be interrogated by various methods. For example, the material may be interrogated via an optical technique such as Raman spectroscopy, which involves illuminating the material using a laser causing the material to emit characteristic fingerprint spectra.

The material sampling and ID device 100 may be formed of a flexible material, such as a flexible polymer, that is compatible with various interrogation methods of interest. It may be a chemically nonreactive polymer. For example, an optically clear plastic such as low-density polyethylene can be used to enable a variety of optical and chemical interrogation techniques. Non-reactive translucent or opaque polymers such as polytetrafluoroethylene may also be used where chemical compatibility concerns exist. For embodiments where an optically opaque material is used for the construction of the sampling device 100, the elongated tube 120 can be detachable and removed to allow for visual inspection of the material of interest for use with colorimetric agents.

FIG. 2 shows the material sampling and identification device 100 supported on a sampling stand 200 for interrogation of a target material 210. The sampling stand 200 is configured/shaped to receive the bulbous end 114 of the sampling device 100, utilizing gravity to keep the target material 210 to be tested contained within the bulbous cavity 112. In the embodiment shown, the sampling stand 200 has a concave interior to receive and support the bulbous end 114 of the sampling device 100. For example, the concave interior of the sampling stand 200 is configured to receive a bottom of the bulbous member 110.

An optical technique such as spatially offset Raman spectroscopy can be utilized to optically interrogate the material of interest through a translucent or opaque sidewall of the sampling device 100. The planar end of the Raman laser probe 220 may be pressed against the planar surface 130 of the bulbous member 110 of the sampling device 100. The bulbous member 110 or elongated tube 120 may include a section of colored material to enable a user to quickly perform visual comparison of the results of colorimetric testing.

FIG. 3 shows the material sampling and identification device 100 supported on the sampling stand 200 for interrogation of the target material 210 using another technique. The target material 210 is contained within the bulbous cavity 112 of the bulbous member 110. A colorimetric reagent may be added to the sampling device 100 via the open end 124 to be mixed with the target material 210 in the bulbous cavity 112.

The sampling stand 200 may be designed for general use or specifically to hold the sampling device 100 to facilitate use of a particular interrogation technique of interest. For example, the sampling stand 200 may be mounted mechanically or magnetically for use inside a Bulk Resolution System such as a spatially offset Raman spectroscopy-based explosives detector. The sampling stand 200 may be configured to shield an end user from any hazardous optical radiation such as laser light. Alternatively, the sampling device 100 may be dimensionally configured to be inserted directly into the vial holder portion of a handheld Raman spectrometer.

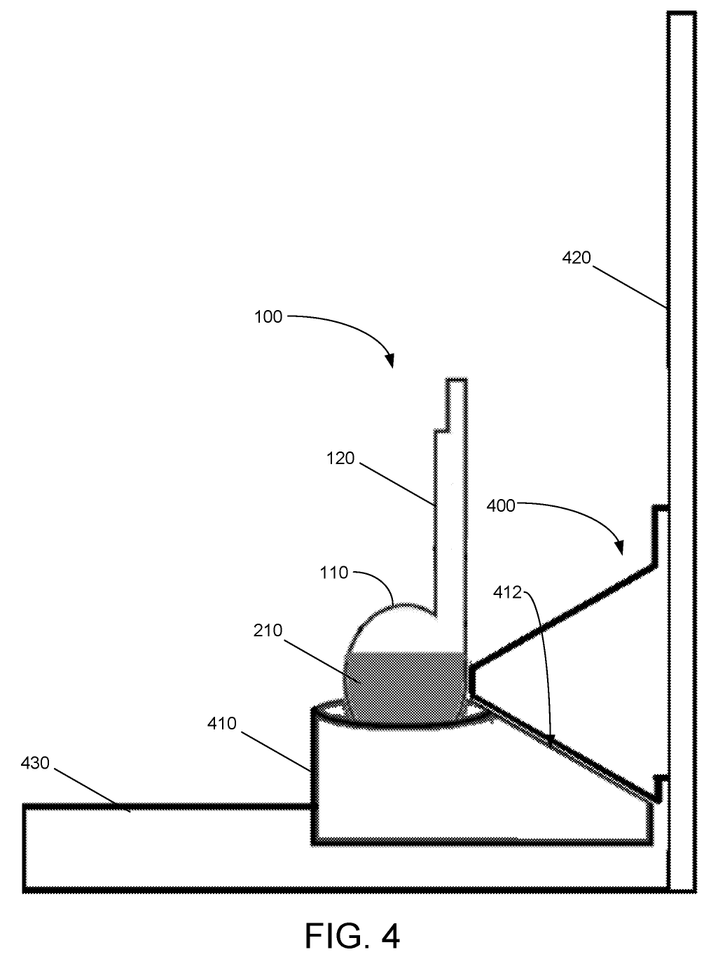

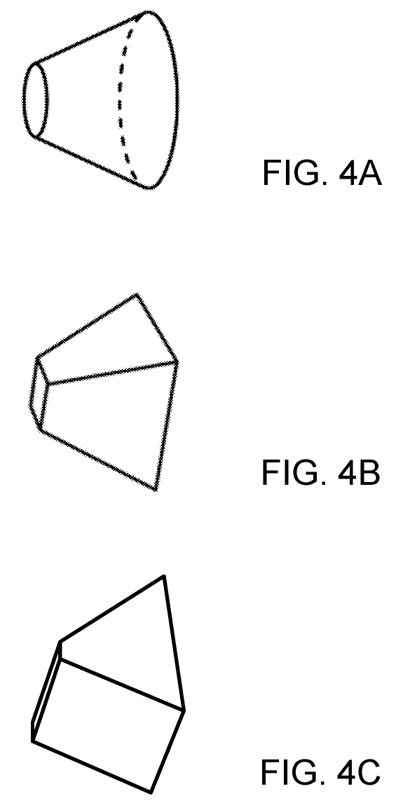

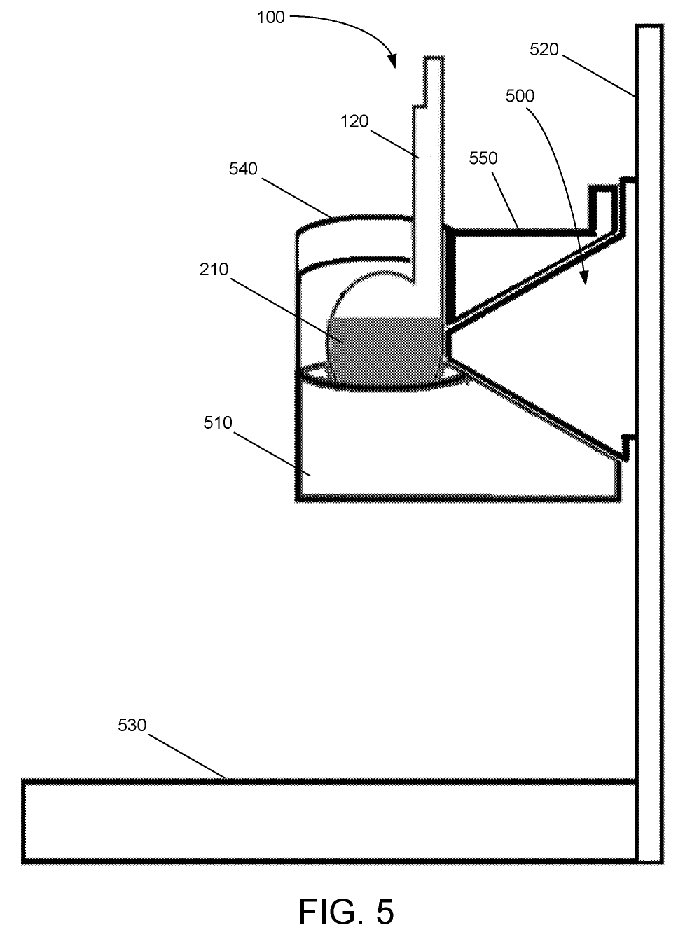

FIG. 4 shows an example of a mounting device 400 for mounting a sampling stand 410 for the sampling device 100. The mounting device includes a wedge mount 400 extending generally horizontally with upper and lower inclined surfaces (e.g., ±20^{o} to ±45^{o}) to a side wall 420 to mount the sampling stand 410. The wedge mount 400 has a shell structure (i.e., not a solid structure) is coupled with or mated with and engages an inclined or sloped surface 412 of the sampling stand 410 (e.g., 20^{o} to 45^{o}) for mounting the sampling stand 410. When mounted, the sampling stand 410 positions the liquid/powder 210 inside the bulb end of the sampling device 100 to be interrogated by laser output. In the first embodiment, the sampling stand 410 includes a bottom that sits on the floor 430 of the interior of a material detection system, such as the Agilent (formerly Cobalt) Spatially Offset Raman Spectroscopy-based or SORS-based detection or screening system.

FIG. 4A shows a blunted cone-shaped mount as an example of a wedge mount. The cone-shaped mount 400 may be configured to align a cone-shaped aperture of a laser probe with the bulbous member 110 to interrogate the target material 210 by laser output from the laser probe. The cone-shaped aperture of the Raman laser probe rests on the sloped portion of the mounting device 400 to ensure that the Raman laser probe is properly aligned with the material 210 inside the sampling device 100.

FIG. 4B shows a blunted pyramid-shaped mount as another example of a wedge mount.

FIG. 4C shows a blunted wedge-shaped mount as another example of a wedge mount.

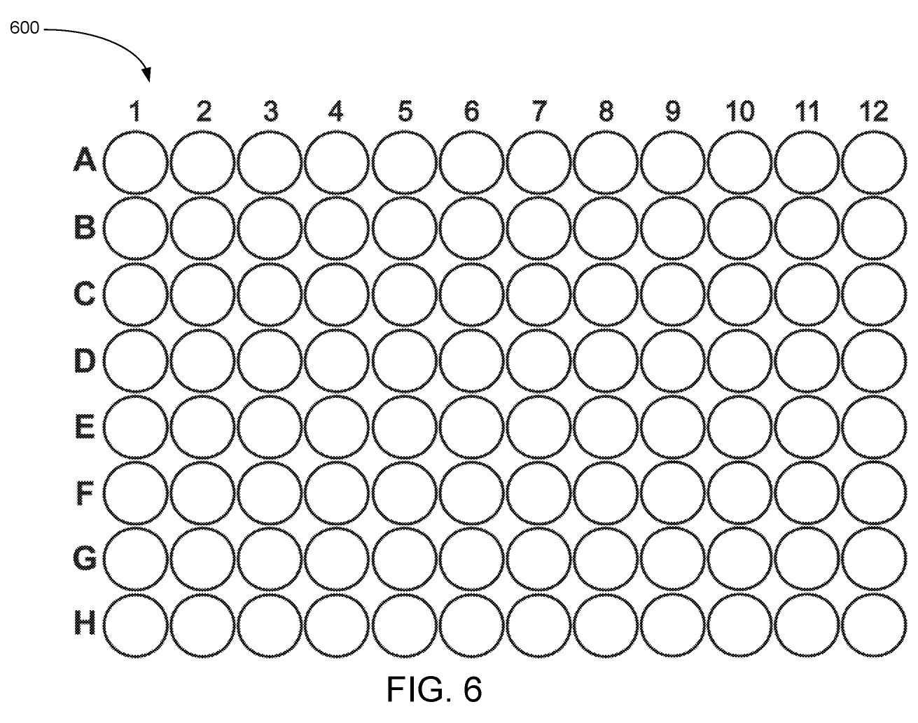

FIG. 5 shows another example of a mounting device 500 for mounting another sampling stand 510 for the sampling device 100. The mounting device includes a cone-shaped mount 500 having a cone-shaped shell structure extending generally horizontally from a side wall 520 to mount the sampling stand 510. When mounted, the sampling stand 510 positions the liquid/powder 210 inside the bulb end of the sampling device 100 to be interrogated by laser output. In the second embodiment, the mounting device 500 is mechanically attached to the cone-shaped aperture at a position above the floor 530 of the interior of the Agilent system. Additional upper supports 540, 550 may be provided to secure and stabilize the sampling device 100. For example, the sampling stand 510 has an upper sampling stand support 540 to provide upper support to the sampling device 100. The upper sampling stand support 540 may be positioned above the bulbous member 110 to provide upper support to the elongated tube 120. The upper mount support 550 is coupled with or mated with and engages the cone-shaped mount 500 and is connected to the upper sampling stand support 540.

The sampling device 100 should be cleaned and/or sterilized prior to packaging in sterile packaging. Embodiments are intended for one-time use to prevent contamination of bulk materials to be sampled. The sampling device 100 and accessories may be packaged as a kit. The packaging for the kit may include obvious labeling to indicate that the sampling device 100 has been sterilized prior to packaging. The packaged kit may further include a containment tray with one or more wells for performing colorimetric testing of the material of interest.

FIG. 6 is a schematic view illustrating an example of a containment tray 600. This tray may or may not be prefilled with colorimetric test reactants.

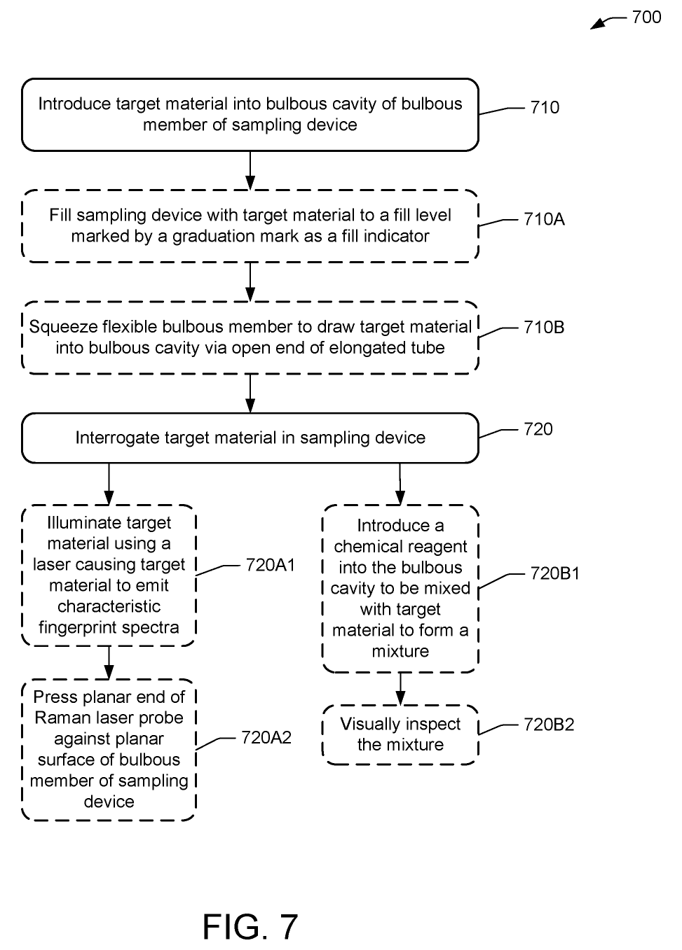

FIG. 7 shows an example of a flow diagram 700 illustrating a method for sampling a target material using the sampling device 100. Step 710 involves introducing the target material 210 via the open end 124 of the elongated tube 120 through the hollow interior 122 into the bulbous cavity 112 of the bulbous member 110 of the sampling device 100. In some embodiments, introducing the target material may include filling the sampling device with the target material to a fill level marked by a graduation mark as a fill indicator 140 on the sampling device (step 710A). In specific embodiments, introducing the target material may include squeezing a flexible bulbous member 110 (e.g., made of a flexible polymer) to draw the target material into the bulbous cavity via the open end of the elongated tube (step 710B).

Step 720 involves interrogating the target material in the sampling device. In some embodiments, interrogating the target material may involve illuminating the target material using a laser causing the target material to emit characteristic fingerprint spectra (step 720A1). It may include pressing a planar end of a Raman laser probe 220 against a planar surface 130 of the bulbous member of the sampling device to optically interrogate the target material in the sampling device (step 720A2). In specific embodiments, interrogating the target material may involve introducing a chemical reagent through the open end of the elongated tube into the bulbous cavity of the bulbous member to be mixed with the target material (step 720B1) and visually inspecting a mixture of the chemical reagent and the target material (step 720B2). The chemical reagent may include a colorimetric agent. Interrogating the target material may further include visually inspecting the mixture via a translucent bulbous member. Alternatively, it may include detaching the elongated tube, which is detachably coupled to the bulbous member, from the bulbous member to expose the mixture, and visually inspecting the exposed mixture.

The inventive concepts taught by way of the examples discussed above are amenable to modification, rearrangement, and embodiment in several ways. Accordingly, although the present disclosure has been described with reference to specific embodiments and examples, persons skilled in the art will recognize that changes may be made in form and detail without departing from the spirit and scope of the disclosure.

An interpretation under 35 U.S.C. §112(f) is desired only where this description and/or the claims use specific terminology historically recognized to invoke the benefit of interpretation, such as “means,” and the structure corresponding to a recited function, to include the equivalents thereof, as permitted to the fullest extent of the law and this written description, may include the disclosure, the accompanying claims, and the drawings, as they would be understood by one of skill in the art.

To the extent the subject matter has been described in language specific to structural features and/or methodological steps, it is to be understood that the subject matter defined in the appended claims is not necessarily limited to the specific features or steps described. Rather, the specific features and steps are disclosed as example forms of implementing the claimed subject matter. To the extent headings are used, they are provided for the convenience of the reader and are not to be taken as limiting or restricting the systems, techniques, approaches, methods, devices to those appearing in any section. Rather, the teachings and disclosures herein can be combined, rearranged, with other portions of this disclosure and the knowledge of one of ordinary skill in the art. It is the intention of this disclosure to encompass and include such variation.

The indication of any elements or steps as “optional” does not indicate that all other or any other elements or steps are mandatory. The claims define the invention and form part of the specification. Limitations from the written description are not to be read into the claims.

What is claimed is:

1. A material sampling and identification device comprising:
a bulbous member including a bulbous cavity at a closed end; and
an elongated tube having a connection end coupled to the bulbous member and including a hollow interior which extends from the bulbous cavity of the bulbous member through the connection end to an open end which is configured as a scoopula end.

2. The device of claim 1,
wherein the connection end of the elongated tube is coupled to the bulbous member at an offset location of the bulbous member to be offset to one side relative to the bulbous member.

3. The device of claim 1,
wherein the bulbous member has a generally round shape with a planar side surface on a lateral side, the planar side surface being parallel to a longitudinal axis of the elongated tube.

4. The device of claim 1,
wherein at least one of the bulbous member or the elongated tube includes one or more graduation marks as one or more fill indicators spaced along a longitudinal axis of the elongated tube.

5. The device of claim 1,
wherein the elongated tube decreases in lateral size from the connection end to the open end along a longitudinal axis.

6. The device of claim 1,
wherein the elongated tube is detachably coupled to the bulbous member.

7. The device of claim 1,
wherein the bulbous member comprises a flexible polymer.

8. A method for sampling a target material using a sampling device which includes a bulbous member having a bulbous cavity at a closed end and an elongated tube having a connection end coupled to the bulbous member and including a hollow interior which extends from the bulbous cavity of the bulbous member through the connection end to an open end which is configured as a scoopula end, the method comprising:
introducing the target material via the open end of the elongated tube through the hollow interior into the bulbous cavity of the bulbous member of the sampling device; and
interrogating the target material in the sampling device.

9. The method of claim 8,
wherein interrogating the target material in the sampling device comprises illuminating the target material using a laser causing the target material to emit characteristic fingerprint spectra.

10. The method of claim 9,
wherein interrogating the target material in the sampling device comprises pressing a planar end of a Raman laser probe against a planar surface of the bulbous member of the sampling device to optically interrogate the target material in the sampling device.

11. The method of claim 8,
wherein interrogating the target material in the sampling device comprises introducing a chemical reagent through the open end of the elongated tube into the bulbous cavity of the bulbous member to be mixed with the target material.

12. The method of claim 11, wherein the chemical reagent comprises a colorimetric agent, the method further comprising:
visually inspecting a mixture of the colorimetric agent and the target material via the bulbous member which is translucent.

13. The method of claim 11, wherein the chemical reagent comprises a colorimetric agent, the method further comprising:
detaching the elongated tube, which is detachably coupled to the bulbous member, from the bulbous member to expose a mixture of the colorimetric agent and the target material; and
visually inspecting the exposed mixture.

14. The method of claim 8,
wherein the bulbous member comprises a flexible polymer; and
wherein introducing the target material comprises squeezing the bulbous member to draw the target material into the bulbous cavity via the open end of the elongated tube.

15. A material sampling and identification system comprising:
a bulbous member including a bulbous cavity at a closed end;
an elongated tube having a connection end coupled to the bulbous member and including a hollow interior which extends from the bulbous cavity of the bulbous member through the connection end to an open end; and
a sampling stand having a concave interior to receive the bulbous member and position the elongated tube above the bulbous member to keep a target material contained within the bulbous cavity.

16. The system of claim 15, further comprising:
a mounting device having a wedge mount configured to extend generally horizontally to mount the sampling stand.

17. The system of claim 16,
wherein the wedge mount comprises a cone-shaped mount configured to align a cone-shaped aperture of a laser probe with the bulbous member to interrogate the target material by laser output from the laser probe.

18. The system of claim 16,
wherein the sampling stand includes an inclined surface; and
wherein the wedge mount is configured to be coupled with the inclined surface of the sampling stand for mounting the sampling stand.

19. The system of claim 18,
wherein the concave interior of the sampling stand is configured to receive a bottom of the bulbous member; and
wherein the sampling stand further includes an upper sampling stand support positioned above the bulbous member to provide upper support to the elongated tube.

20. The system of claim 19, further comprising:
an upper mount support to be coupled with the wedge mount and to be connected to the upper sampling stand support.
